(19) United States
(12) Patent Application Publication
(10) Pub. No.: US 2020/0297617 A1
(43) Pub. Date: Sep. 24, 2020

(54) COMPOSITION FOR SKIN EXTERNAL USE FOR REDUCING WRINKLE AND COSMETIC PRODUCT

(71) Applicant: Guangdong Bawei Biotechnology Co., Ltd., Guangdong (CN)

(72) Inventors: Ruixue Liu, Guangdong (CN); Qunying Leng, Guangdong (CN); Qinyuan Leng, Guangdong (CN)

(73) Assignee: Guangdong Bawei Biotechnology Co., Ltd., Guangdong (CN)

(21) Appl. No.: 16/430,063

(22) Filed: Jun. 3, 2019

(30) Foreign Application Priority Data
Mar. 19, 2019 (CN) 201910208848.6

(51) Int. Cl. A61K 8/9789; A61K 8/9728; A61Q 19/08; A61K 8/9794

(52) U.S. Cl. CPC A61K 8/9789; A61K 8/9728; A61K 2800/82; A61K 8/9794; A61Q 19/08

(57) ABSTRACT

The present disclosure provides a composition for skin external use for reducing wrinkle and a cosmetic product, which relates to the field of cosmetic products. The composition for skin external use for reducing wrinkle provided in the present disclosure includes, in parts by weight, 14-23 parts of an Anogeissus extract, 9-15 parts of a Punica extract, 4-8 parts of a Palmae extract and 3-6 parts of a polygonatum odoratum extract. The cosmetic product provided in the present disclosure includes the composition for skin external use for reducing wrinkle. The composition for skin external use for reducing wrinkle and the cosmetic product provided in the present disclosure solve the problem of poor compatibility between the effective components of the extracts, reduce skin wrinkles by inhibiting the formation of reactive oxide species, inhibiting the MMPs activity and improving collagen synthesis, and achieve more remarkable anti-aging effects.

### CROSS-REFERENCE TO RELATED APPLICATION

The present disclosure claims the priority to a Chinese Patent Application No. 201910208848.6, filed with the National Intellectual Property Administration, PRC on Mar. 19, 2019 and entitled “Composition for Skin External Use for Reducing Wrinkle and Cosmetic Product”, the content of which is incorporated herein by reference in entirety.

### TECHNICAL FIELD

The present disclosure relates to the field of cosmetic product, and particularly to a composition for skin external use for reducing wrinkle and a cosmetic product.

### BACKGROUND ART

The dermis is a dense network of fibers and elastic tissues, and is composed of a large number of fibrin such as collagens and elastins, hyaluronic acid serves as a liquid matrix, and the three are all filled in the extracellular matrix with each other. As age increases, the synthesis of collagens and elastins in fibroblasts decreases. Moreover, matrix metalloproteinases (MMPs) degrade collagens and elastins in the dermis, and the endogenous free radicals also promote the cross linking of collagens, resulting in the decrease of skin tension. Further, the free radicals also directly degrade hyaluronic acid, so that the water-retaining capacity and supporting power of the dermis are reduced.

In order to reduce skin wrinkles, people try to slow aging in different ways, such as increasing collagens and elastins in skin, inhibiting MMPs activity, and reducing free radicals in the body. As there is a growing demand for natural, low side effect and effective products on the market, it has become a trend on the market to use extracts or extract compositions with anti-aging properties, instead of traditional chemicals, as skin care additives. The existing research solves the problem of skin aging in a relatively single way. In addition, due to the relatively low content of effective components that actually work in many plant extracts or the poor compatibility of the effective components in the extracts with other compositions, poor efficacy is achieved when the entire composition is added to cosmetic products.

In view of this, the present disclosure is proposed.

### SUMMARY

The present disclosure provides a composition for skin external use for reducing wrinkle, comprising, in parts by weight, 14-23 parts of an Anogeissus extract, 9-15 parts of a Punica extract, 4-8 parts of a Palmae extract and 3-6 parts of a polygonatum odoratum extract.

The present disclosure further provides a cosmetic product, comprising the composition for skin external use for reducing wrinkle.

### DETAILED DESCRIPTION OF EMBODIMENTS

The embodiments of the present disclosure will be described below in detail in connection with the examples, but it will be understood by those skilled in the art that the examples below are only used for illustrating the present disclosure, but should not be considered as limiting the scope of the present disclosure. Examples are carried out in accordance with conventional conditions or conditions recommended by the manufacturer if no specific conditions are specified in the examples. Reagents or instruments used, whose manufacturers are not specified, are all conventional products that are available commercially.

It should be particularly noted that the euterpe oleracea fruit extract and the Punica granatum extract used in the present disclosure were purchased from Guangzhou Ruiyu Chemical Technology Co., Ltd., the polygonatum odoratum extract was purchased from LB Cosmeceutical Technology (Shanghai) Co., Ltd., and the dictyophora indusiata (mushroom) extract was purchased from Nutri-woods Holdings Corporation. The Anogeissus extracts, except those self-made, were purchased from Guangzhou Ownsnow Chemical Co., Ltd.

Embodiments of the present disclosure provide a composition for skin external use for reducing wrinkle, which reduces skin wrinkles by the synergistic effects of three ways, i.e., inhibiting the formation of reactive oxide species, inhibiting the MMPs activity and improving collagen synthesis, and solves the problem of poor compatibility between the effective components of the extracts by rationally designing the components and proportioning of the components of the composition, so as to achieve better and more remarkable anti-aging effects.

Embodiments of the present disclosure further provide a cosmetic product, in which the composition for skin external use for reducing wrinkle is used as an active component, which in turn provides a wide range of application scenarios to the use of the cosmetic product.

The Anogeissus extract has the effects of inhibiting the MMPs activity and inhibiting the formation of free radicals, etc., these effects are mainly based on the effective components such as 3,3′-O-dimethylellagic acid and ellagic acid contained in the Anogeissus extract. The applicant has found that at the time of using the Anogeissus extract, the addition of the extract to the product is restricted due to the problem of water solubility of the effective components of the extract, thereby hindering the active components thereof from playing a better role; when the Anogeissus extract is used in combination with the Punica extract, the water solubility of the Anogeissus extract can be improved, which leads to wider application thereof and better effects in the products; when added to the composition, the Palmae extract works synergistically with the Anogeissus extract to improve effect, to prevent collagen degradation; and when added to the composition, the polygonatum odoratum extract improves the anti-free radical effect of the Anogeissus extract.

In an optional embodiment, the Anogeissus extract may be in 14 parts by weight, 15 parts by weight, 16 parts by weight, 17 parts by weight, 18 parts by weight, 19 parts by weight, 20 parts by weight, 21 parts by weight, 22 parts by weight, 23 parts by weight, and any parts by weight in 14-23 parts by weight; the Punica extract may be in 9 parts by weight, 10 parts by weight, 11 parts by weight, 12 parts by weight, 13 parts by weight, 14 parts by weight, 15 parts by weight, and any parts by weight in 9-15 parts by weight; the Palmae extract may be in 4 parts by weight, 5 parts by weight, 6 parts by weight, 7 parts by weight, 8 parts by weight and any parts by weight in 4-8 parts by weight; and the polygonatum odoratum extract may be in 3 parts by weight, 4 parts by weight, 5 parts by weight, 6 parts by weight, and any parts by weight in 3-6 parts by weight.

Preferably, the Anogeissus extract is an Anogeissus leiocarpus bark extract, the Punica extract is a Punica granatum extract, and the Palmae extract is a euterpe oleracea fruit extract.

Optimizing the extracts is helpful to the optimization and improvement of the performance of the composition itself, so that the effects thereof in inhibiting the MMPs activity, inhibiting the formation of free radicals and improving collagen synthesis are further improved, the water solubility is better and the synergistic effect is stronger.

Further preferably, the composition for skin external use for reducing wrinkle further comprises, in parts by weight, 1-5 parts of a dictyophora indusiata (mushroom) extract.

The dictyophora indusiata (mushroom) extract is rich in polysaccharides, proteins, amino acids, vitamins and a variety of minerals, and can help promote collagen production.

In an optional embodiment, the dictyophora indusiata (mushroom) extract may be in 1 part by weight, 2 parts by weight, 3 parts by weight, 4 parts by weight, 5 parts by weight and any parts by weight in 1-5 parts by weight.

More preferably, the composition for skin external use for reducing wrinkle comprises, in parts by weight, 18-20 parts of the Anogeissus leiocarpus bark extract, 11-14 parts of the Punica granatum extract, 5-7 parts of the euterpe oleracea fruit extract, 4-5 parts of the polygonatum odoratum extract, and 2-4 parts of the dictyophora indusiata (mushroom) extract.

By further optimizing the amount of each component, a composition for skin external use for reducing wrinkle with better effects can be obtained.

Preferably, a method for preparing the Anogeissus extract comprises the steps of:

pulverizing the Anogeissus bark and then sieving the same, then soaking the same by adding a first solvent; and performing ultrasonic extraction to the soaked mixture to obtain an extraction solution; and

filtering the extraction solution to obtain a filtrate, adding a second solvent to the filtrate, stirring and cooling the same, crystallizing to obtain a precipitate, and then drying the same to obtain the Anogeissus extract.

By optimizing the extraction process of the Anogeissus extract, it is possible to improve the content of the effective components in the obtained Anogeissus extract, and reduce the loss of the effective components in the extraction process, thereby ensuring the overall anti-aging effect of the composition.

Before pulverization, the Anogeissus bark is preferably washed and dried.

Preferably, the sieve used for the sieving has 70-100 meshes; the first solvent is dimethyl sulfoxide or N-methylpyrrolidone, the soaking time is 1-2.5 h, and the material-liquid ratio is 1:10-15 g/mL.

The limitation on the mesh number of the sieve serves to ensure that the particle size of the material obtained by pulverization is kept in an optimal range so as to facilitate the subsequent soaking and extraction. In order to ensure the extraction effect, the first solvent is optimized, and such optimization is to obtain suitable solvents, soaking time and material-liquid ratio by experiments based on the inventor's knowledge about the components of the bark of the Anogeissus plant and the analysis of the polarity of each component and various solvents. Most preferably, the first solvent is dimethyl sulfoxide.

Preferably, the extraction temperature for the ultrasonic extraction is 70-90° C., the ultrasonic power is 250-450 W, and the extraction time is 15-35 min.

The selection for the extraction temperature, ultrasonic power and extraction time is made in consideration of the dissolution rate and solubility of the effective components in the solvents during ultrasonic extraction on the one hand, and on the other hand in consideration of the stability of the effective components at the temperature to avoid damaging the structure thereof.

Preferably, the second solvent is one of methanol, ethanol and water, and the volume ratio of the second solvent to the filtrate is 1:2-5.

The selection for the second solvent is mainly made in consideration of the solubility of the effective components therein and the operability of the cooling and crystallization process.

Optionally, the cooling rate is 2-4° C./s, and the temperature of the crystallization and precipitation is 10-20° C.

The control of the cooling rate and the temperature of the crystallization and precipitation mainly serves to ensure the purity of the extract.

Since the water solubility of the effective components in the composition for skin external use for reducing wrinkle provided in the present disclosure is enhanced, the effective components can be used as cosmetic active components in the products of various dosage forms such as toner, essence, emulsion, cream, facial mask and gel. The weight proportion of the composition for skin external use for reducing wrinkle provided in the present disclosure in the whole cosmetic product is preferably 0.01-5.2%. If the content of the composition is lower than 0.01%, no significant effect can be achieved, so that they cannot be used as active components; and if the content of the composition is higher than 5.2%, there will occur a problem in the stability of the dosage form.

### EXAMPLE 1

A composition for skin external use for reducing wrinkle was prepared by taking, in parts by weight, 14 parts of the Anogeissus extract, 11 parts of the Punica extract, 4 parts of the Palmae extract, and 6 parts of the polygonatum odoratum extract.

### EXAMPLE 2

A composition for skin external use for reducing wrinkle was prepared by taking, in parts by weight, 18 parts of the Anogeissus leiocarpus bark extract, 9 parts of the Punica granatum extract, 8 parts of the euterpe oleracea fruit extract, and 3 parts of the polygonatum odoratum extract.

### EXAMPLE 3

A composition for skin external use for reducing wrinkle was prepared by taking, in parts by weight, 23 parts of the Anogeissus leiocarpus bark extract, 15 parts of the Punica granatum extract, 5 parts of the euterpe oleracea fruit extract, and 4 parts of the polygonatum odoratum extract.

### EXAMPLE 4

A composition for skin external use for reducing wrinkle was prepared by taking, in parts by weight, 18 parts of the Anogeissus leiocarpus bark extract, 11 parts of the Punica granatum extract, 7 parts of the euterpe oleracea fruit extract, 5 parts of the polygonatum odoratum extract, and 1 part of the dictyophora indusiata (mushroom) extract.

### EXAMPLE 5

A composition for skin external use for reducing wrinkle was prepared by taking, in parts by weight, 20 parts of the Anogeissus leiocarpus bark extract, 14 parts of the Punica granatum extract, 6 parts of the euterpe oleracea fruit extract, 4 parts of the polygonatum odoratum extract, and 5 parts of the dictyophora indusiata (mushroom) extract.

The Anogeissus extracts used in Examples 1-5 were all purchased from Guangzhou Ownsnow Chemical Co., Ltd.

### EXAMPLE 6

A composition for skin external use for reducing wrinkle was prepared by taking, in parts by weight, 19 parts of the Anogeissus leiocarpus bark extract, 12 parts of the Punica granatum extract, 6 parts of the euterpe oleracea fruit extract, 4.5 parts of the polygonatum odoratum extract, and 3 parts of the dictyophora indusiata (mushroom) extract.

In the above, a method for preparing the Anogeissus leiocarpus bark extract comprises the steps of:

pulverizing the Anogeissus leiocarpus bark and then sieving the same with a 70-mesh sieve, and then soaking the same for 1 h by adding a first solvent, dimethyl sulfoxide at a material-liquid ratio of 1:10 g/mL;

performing ultrasonic extraction to the soaked mixture to obtain an extraction solution, with an extraction temperature of 70° C., an ultrasonic power of 250 W, and an extraction time of 15 min;

filtering the extraction solution to obtain a filtrate, and adding a second solvent, methanol to the filtrate, with the volume ratio of the second solvent to the filtrate of 1:2; and

stirring and cooling the same, with the cooling rate controlled at 2° C./s and the temperature of the crystallization and precipitation controlled at 20° C., filtering and centrifuging the same after crystallization and precipitation to obtain a precipitate, washing the precipitate twice with ethanol aqueous solution with volume fraction of 40%, and then vacuum drying the same to obtain the Anogeissus leiocarpus bark extract.

### EXAMPLE 7

A composition for skin external use for reducing wrinkle was prepared by taking, in parts by weight, 18.5 parts of the Anogeissus leiocarpus bark extract, 13 parts of the Punica granatum extract, 6.5 parts of the euterpe oleracea fruit extract, 4 parts of the polygonatum odoratum extract, and 2 parts of the dictyophora indusiata (mushroom) extract.

In the above, a method for preparing the Anogeissus leiocarpus bark extract comprises the steps of:

pulverizing the Anogeissus leiocarpus bark and then sieving the same with a 80-mesh sieve, and then soaking the same for 2 h by adding a first solvent, dimethyl sulfoxide at a material-liquid ratio of 1:12 g/mL;

performing ultrasonic extraction to the soaked mixture to obtain an extraction solution, with an extraction temperature of 80° C., an ultrasonic power of 300 W, and an extraction time of 25 min;

filtering the extraction solution to obtain a filtrate, and adding a second solvent, water to the filtrate, with the volume ratio of the second solvent to the filtrate of 1:4; and

stirring and cooling the same, with the cooling rate controlled at 3° C./s and the temperature of the crystallization and precipitation controlled at 15° C., filtering and centrifuging the same after crystallization and precipitation to obtain a precipitate, washing the precipitate twice with ethanol aqueous solution with volume fraction of 40%, and then vacuum drying the same to obtain the Anogeissus leiocarpus bark extract.

### EXAMPLE 8

The composition in this example is the same as that in Example 3, except that the Anogeissus leiocarpus extract is prepared as follows:

washing, drying and pulverizing the Anogeissus leiocarpus bark and then sieving the same with a 100-mesh sieve, and then soaking the same for 2.5 h by adding a first solvent, N-methylpyrrolidone at a material-liquid ratio of 1:15 g/mL;

performing ultrasonic extraction to the soaked mixture to obtain an extraction solution, with an extraction temperature of 90° C., an ultrasonic power of 450 W, and an extraction time of 35 min;

filtering the extraction solution to obtain a filtrate, and adding a second solvent, ethanol to the filtrate, with the volume ratio of the second solvent to the filtrate of 1:5; and

stirring and cooling the same, with the cooling rate controlled at 4° C./s and the temperature of the crystallization and precipitation controlled at 10° C., filtering and centrifuging the same after crystallization and precipitation to obtain a precipitate, washing the precipitate three times with ethanol aqueous solution with volume fraction of 40%, and then vacuum drying the same to obtain the Anogeissus leiocarpus bark extract.

The composition for skin external use for reducing wrinkle provided in the present disclosure can be used as a skin care additive and added to cosmetic products of various dosage forms such as toner, essence, emulsion, cream, facial mask and gel, etc.

### CONTRAST EXAMPLE 1

A composition, mainly comprising, in parts by weight, 14 parts of the Anogeissus leiocarpus bark extract, 4 parts of the euterpe oleracea fruit extract, and 6 parts of the polygonatum odoratum extract.

### CONTRAST EXAMPLE 2

A composition, mainly comprising, in parts by weight, 14 parts of the Anogeissus leiocarpus bark extract, 11 parts of the Punica granatum extract, and 6 parts of the polygonatum odoratum extract.

### CONTRAST EXAMPLE 3

A composition, mainly comprising, in parts by weight, 14 parts of the Anogeissus leiocarpus bark extract, 11 parts of the Punica granatum extract, and 4 parts of the euterpe oleracea fruit extract.

The compositions obtained in Examples 1-8 and Contrast examples 1-3 were subjected to performance tests.

Test 1: Inhibition of the Production of Active Oxygen

For fluorescence measurement, 100 μL of human keratinocyte HaCaT cell strains were added to each well of a 96-well blackboard at a density of 2.0×105 cells/mL, and cultured for 24 h in an incubator with 5% carbon dioxide at 37° C., and the cells were then treated with the compositions of Examples 1-8 and Contrast examples 1-3, the compositions accounting for 0.05% of the total weight. After 24 h of culture of the cells together with the compositions of these examples and contrast examples, each well was washed with HCSS (HEPES-buffered control salt solution) to remove the remaining mediums, and 100 μL of a 20 μM DCFH-DA (2′,7′-dichlorodihydro-fluorescein diacetate) HCSS solution was added to each well. The cells were then cultured for 20 min in the incubator with 5% carbon dioxide at 37° C. and then washed with HCSS. Then 100 μL of HCSS solution, containing compositions of different examples and contrast examples, was added to each well and each well was irradiated with UV-B light (30 mJ/cm2). After 3 h, the fluorescence intensity per well was measured with a fluorometer (Ex=485 nm, Em=530 nm), and measurement was repeated 6 times and the mean value of the measurement results was taken. Here, the control group was a group that was not treated with the compositions provided in the examples and contrast examples.

The measurement results are shown in Table 1:

TABLE 1
Inhibitory effects on active oxygen

| | proportions relative to |
|---|---|
| test material | the control group (%) |
| control group | 100 |
| control group + ultraviolet radiation | 186.4 |
| Example 1 + ultraviolet radiation | 142.1 |
| Example 2 + ultraviolet radiation | 140.3 |
| Example 3 + ultraviolet radiation | 139.4 |
| Example 4 + ultraviolet radiation | 135.6 |
| Example 5 + ultraviolet radiation | 128.3 |
| Example 6 + ultraviolet radiation | 114.8 |
| Example 7 + ultraviolet radiation | 115.6 |
| Example 8 + ultraviolet radiation | 119.4 |
| Contrast example 1 + ultraviolet radiation | 165.4 |
| Contrast example 2 + ultraviolet radiation | 166.2 |
| Contrast example 3 + ultraviolet radiation | 171.7 |

As can be seen from the results of Table 1 above, after the cells were irradiated with ultraviolet rays, the level of intracellular active oxygen was increased to 186.4%, as compared with the cells in the control group without ultraviolet irradiation. When the cells were treated with the composition for skin external use for reducing wrinkle provided in the present disclosure, the content of intracellular active oxygen was limited. In particular, after the addition of the Anogeissus leiocarpus bark extract prepared by the preparation method of the present disclosure, the inhibition of the active oxygen of the whole composition reached 114.8%, as compare with the control group. The compositions of the examples showed more pronounced inhibition effect on active oxygen generation than the compositions of the contrast examples.

Test 2: Analysis of Inhibition of MMP-1 Expression

In order to evaluate the effect on reducing wrinkles of the composition, it was experimentally demonstrated that the composition was capable of inhibiting the expression of MMP-1. Human fibrocytes (HS68 cells, purchased from ATCC (Rockville, Md., USA)) were placed in a well plate at a density of 7.5×104 cells/well, and incubated in DMEM containing 10% FBS for 24 h, and then washed with PBS (phosphate buffer), followed by the addition of 0.5 ml of PBS therein, the resultant incubated cells were irradiated with ultraviolet rays (UVB30 mJ/cm2) to induce the expression of MMP-1. After the replacement with serum-free DMEM, the resultant product was treated with the compositions (having a mass concentration of 1%) of the examples and the contrast examples. The amount of supernatant obtained after 48 h incubation was determined using an MMP-1 ELISA kit. The human dermal fibroblasts HS68 were treated with ultraviolet rays to increase the expression of MMP-1, and then the resultant HS68 cells were treated with each sample to study whether the expression of MMP-1 was decreased. In the above, retinoic acid (RA, 5 μM, which is known to inhibit the expression of MMP-1) was used as a positive control group. The test results were shown in Table 2.

TABLE 2
Effect on inhibition of MMP-1 expression

| | proportions relative to |
|---|---|
| test material | the control group (%) |
| control group | 100 |
| control group + ultraviolet radiation | 194 |
| Example 1 + ultraviolet radiation | 143 |
| Example 2 + ultraviolet radiation | 147 |
| Example 3 + ultraviolet radiation | 140 |
| Example 4 + ultraviolet radiation | 132 |
| Example 5 + ultraviolet radiation | 136 |
| Example 6 + ultraviolet radiation | 122 |
| Example 7 + ultraviolet radiation | 123 |
| Example 8 + ultraviolet radiation | 124 |
| Contrast example 1 + ultraviolet radiation | 178 |
| Contrast example 2 + ultraviolet radiation | 180 |
| Contrast example 3 + ultraviolet radiation | 174 |
| RA + ultraviolet radiation | 118 |

As can be seen from the experimental results in Table 2, the compositions of Examples 1-8 had more remarkable effect on inhibiting MMP-1 expression than the compositions of the contrast examples, especially after the addition of the Anogeissus leiocarpus bark extract obtained by the preparation method of the present disclosure, the effect on inhibiting MMP-1 expression of the present disclosure is similar to that of retinoic acid.

Test 3: Effect on Collagen Biosynthesis of Human Skin Cells

Human fibroblasts were cultured in a 24-well microculture plate, and their medium was changed to a medium containing 1.5% by mass of the compositions of the examples and the contrast examples. On day 3 of the culture, 0.5 mL of DMEM medium containing 10% fetal bovine serum (FBS) was added to each well, followed by the addition of 10 μCi of L[2,3,4,5-3H]-proline therein. 24 h later, the medium and the cells contained in each well were tilted and 5% trichloroacetic acid solution was added thereto. Each sample was then divided into 2 test tubes, and 1 unit/μL of type I collagenase was added to one of each pair of test tubes, followed by culturing at 37° C. for 90 min; and the other test tube was stored at 4° C. 0.05 ml of 50% TCA was then added to each test tube, the test tube was placed at 4° C. for 20 min, and each test tube was centrifuged at 12000 rpm for 10 min. Each set of supernatant and precipitate was treated with a liquid scintillation counter to obtain a CPM value, and then the relative collagen synthesis value (RCB) was calculated for the examples and the control group based on the following mathematical formula I. The result obtained was a relative value, with the value of the control group in which the medium contained no composition being set to 100. The test results were shown in Table 3.

$$RCB ( \% ) = \frac{collagen cpm}{total collagen cpm \times 5.4 + collagen cpm} \times 100 \qquad Formula I$$

TABLE 3
Effect of the composition on collagen biosynthesis

| | relative collagen biosynthesis value |
|---|---|
| test material | (RCB) (%) |
| control group | 100 |
| Example 1 | 137 |
| Example 2 | 132 |
| Example 3 | 135 |
| Example 4 | 146 |
| Example 5 | 144 |
| Example 6 | 154 |
| Example 7 | 155 |
| Example 8 | 159 |
| Contrast example 1 | 112 |
| Contrast example 2 | 109 |
| Contrast example 3 | 114 |

As can be seen from the experimental results of Table 3, the compositions of the examples have better effect on promoting collagen biosynthesis than the compositions of the contrast examples.

The compositions provided in Examples 1-8 and Contrast examples 1-3 were prepared into eye creams, the main components (weight percentage) of which were shown in Table 4 below:

TABLE 4
Eye cream components table

| phase | INCI name | content/% |
|---|---|---|
| A phase | C14-22 alcohols | 1.5 |
| | glyceryl stearate | 1.6 |
| | cetearyl alcohol | 2.4 |
| | squalane | 2.5 |
| | dimethicone | 2.5 |
| | methylparaben | 0.12 |
| | caprylic acid | 0.13 |
| B phase | xanthan gum | 0.13 |
| | glycerin | 2 |
| | PEG-8 | 6 |
| | water | 78.32 |
| C phase | dimethiconol | 0.3 |
| | polyacrylamide | 0.5 |
| D phase | compositions of | 2 |
| | examples and contrast | |
| | examples | |

Test 4: Skin Elasticity Test

1) Experiment method: 40 female and 20 male volunteers, aged 30-45, having healthy skin without injuries were selected. The volunteers were randomly divided into 12 groups, with 5 volunteers in each group, eye creams containing the compositions of Examples 1-8 and Contrast examples 1-3 (see Table 4) were applied to these groups of volunteers, respectively, and at the same time there was set a control group in which the volunteers were treated with an eye cream without the compositions of the examples and the contrast examples. After cleaning the face in the morning and evening, the subjects applied a suitable amount (the size of a soybean) of eye cream evenly around the eyes, and used the eye cream continuously for 4 weeks. During the experiment, the subjects were not allowed to apply any other cosmetic products to the experiment site.

2) Experiment instruments

The skin elasticity tester CutometerMPA580 Host and the elastic fiber tissue testing probe Reviscometer RV600 (CK Co., Germany) were used.

3) Elasticity testing method: the skin conditions at week 0 and week 4 were tested respectively using the skin elasticity tester Cutometer MPA580. The test site was the outer corners of the eyes. At the time of measurement, the same measurement area was measured 3 times by the probe, and the average value was taken. The test parameter is R2 (the ratio of the rebound amount Ua of the skin in the absence of negative pressure to the maximum stretching amount Uf in the presence of negative pressure), the closer to 1 R2 is, the better the elasticity of the skin is, and the average change rate of R2=the average change value of R2/R2 value at week 0. The test parameter R5 represents the ratio of the elastic portion of the skin recovery process to the elastic portion of the negative pressure application process during the first cycle of the skin test, and the closer to 1 this value is, the better the skin elasticity is. The average change rate of R5=the average change value of R5/R5 value at week 0. The specific test results of skin elasticity were shown in Table 5.

TABLE 5
Test results of skin elasticity

| | skin elasticity R2 | | | skin elasticity R5 | | |
|---|---|---|---|---|---|---|
| | | | average | | | average |
| | | | change | | | change |
| samples | week 0 | week 4 | rate (%) | week 0 | week 4 | rate (%) |
| Example 1 | 0.532 | 0.618 | 16.1 | 0.714 | 0.794 | 11.2 |
| Example 2 | 0.604 | 0.709 | 17.4 | 0.723 | 0.813 | 12.4 |
| Example 3 | 0.637 | 0.757 | 18.9 | 0.697 | 0.769 | 10.3 |
| Example 4 | 0.554 | 0.668 | 20.5 | 0.707 | 0.807 | 14.1 |
| Example 5 | 0.569 | 0.692 | 21.7 | 0.719 | 0.818 | 13.7 |
| Example 6 | 0.618 | 0.770 | 24.6 | 0.725 | 0.852 | 17.5 |
| Example 7 | 0.612 | 0.759 | 24.0 | 0.696 | 0.821 | 18.0 |
| Example 8 | 0.609 | 0.752 | 23.4 | 0.688 | 0.816 | 18.6 |
| Contrast | 0.514 | 0.536 | 4.3 | 0.701 | 0.723 | 3.2 |
| example 1 | | | | | | |
| Contrast | 0.543 | 0.579 | 6.6 | 0.715 | 0.750 | 4.9 |
| example 2 | | | | | | |
| Contrast | 0.674 | 0.690 | 2.4 | 0.691 | 0.703 | 1.7 |
| example 3 | | | | | | |
| Control | 0.557 | 0.566 | 1.6 | 0.727 | 0.731 | 0.6 |
| group | | | | | | |

As can be seen from the data in Table 5, the elasticity of the skin was effectively improved after the use of the eye cream containing the compositions of Examples 1-8 for a period of time, indicating that the composition of the present disclosure has superior effects on reducing wrinkle and slowing aging.

In the present disclosure, by reasonable matching and scientific proportioning of the Anogeissus bark extract, the Punica granatum extract, the euterpe oleracea fruit extract and the polygonatum odoratum extract and by the synergistic effects of the four components, synergistic effects are respectively achieved on three aspects, i.e., inhibiting the formation of reactive oxide species, inhibiting the MMPs activity and improving collagen synthesis, thereby reducing skin wrinkles in multiple ways. In the present disclosure, by scientific matching the Punica granatum extract and the Anogeissus bark extract, the water solubility of ellagic acid in the Anogeissus bark extract is improved, which leads to wider application of the composition in the products. In the extraction process of the present disclosure, by selecting dimethyl sulfoxide and N-methylpyrrolidone as the first solvent and by designing reasonable process conditions such as the ratio of the second solvent to the filtrate, the cooling rate and the crystallization temperature, the Anogeissus bark extract is enabled to have a higher content of the effective component, ellagic acid, such that when it is applied to a product, more remarkable anti-aging effect is achieved.

Compared with the prior art, the advantageous effects of the present disclosure are as follows:

(1) by means of the synergistic effects of the four components, i.e., the Anogeissus extract, the Punica extract, the Palmae extract and the polygonatum odoratum extract, the composition achieves a synergistic effect on the each of three aspects, i.e., inhibiting the formation of reactive oxide species, inhibiting the MMPs activity and improving collagen synthesis, reduces skin wrinkles in multiple ways, and has remarkable anti-aging effects;

(2) by means of the combined use of the Anogeissus extract and the Punica extract, the water solubility of ellagic acid in the Anogeissus extract is improved, so that the composition has better effect and has wider application; and

(3) by using the composition for skin external use for reducing wrinkle in cosmetic products, various forms of applications may be realized.

Although the present disclosure has been illustrated and described with specific examples, it should be appreciated that many other changes and modifications may be made without departing from the spirit and scope of the present disclosure. It is therefore meant that all such changes and modifications as falling within the scope of the present disclosure are included in the appended claims.

1. A composition for skin external use for reducing wrinkle, comprising, in parts by weight, 14-23 parts of an Anogeissus extract, 9-15 parts of a Punica extract, 4-8 parts of a Palmae extract and 3-6 parts of a polygonatum odoratum extract.

2. The composition for skin external use for reducing wrinkle according to claim 1, wherein the Anogeissus extract is an Anogeissus leiocarpus bark extract, the Punica extract is a Punica granatum extract, and the Palmae extract is a euterpe oleracea fruit extract.

3. The composition for skin external use for reducing wrinkle according to claim 2, further comprising, in parts by weight, 1-5 parts of a dictyophora indusiata extract.

4. The composition for skin external use for reducing wrinkle according to claim 3, comprising, in parts by weight, 18-20 parts of the Anogeissus leiocarpus bark extract, 11-14 parts of the Punica granatum extract, 5-7 parts of the euterpe oleracea fruit extract, 4-5 parts of the polygonatum odoratum extract, and 2-4 parts of the dictyophora indusiata extract.

5. The composition for skin external use for reducing wrinkle according to claim 1, wherein a method for preparing the Anogeissus extract comprises steps of:
pulverizing a Anogeissus bark and then sieving the same, then soaking the same by adding a first solvent; and performing ultrasonic extraction to a soaked mixture to obtain an extraction solution; and
filtering the extraction solution to obtain a filtrate, adding a second solvent to the filtrate, stirring and cooling the same, performing crystallization and precipitation to obtain a precipitate, and then drying the same to obtain the Anogeissus extract.

6. The composition for skin external use for reducing wrinkle according to claim 5, wherein a sieve used for the sieving has 70-100 meshes; the first solvent is dimethyl sulfoxide or N-methylpyrrolidone, a soaking time is 1-2.5 h, and a material-liquid ratio is 1:10-15 g/mL.

7. The composition for skin external use for reducing wrinkle according to claim 5, wherein an extraction temperature for the ultrasonic extraction is 70-90° C., an ultrasonic power is 250-450 W, and an extraction time is 15-35 min.

8. The composition for skin external use for reducing wrinkle according to claim 5, wherein the second solvent is one of methanol, ethanol and water, and a volume ratio of the second solvent to the filtrate is 1:2-5.

9. The composition for skin external use for reducing wrinkle according to claim 5, wherein a cooling rate is 2-4° C./s, and temperature of the crystallization and precipitation is 10-20° C.

10. The composition for skin external use for reducing wrinkle according to claim 6, wherein a cooling rate is 2-4° C./s, and temperature of the crystallization and precipitation is 10-20° C.

11. The composition for skin external use for reducing wrinkle according to claim 7, wherein a cooling rate is 2-4° C./s, and temperature of the crystallization and precipitation is 10-20° C.

12. A cosmetic product, comprising the composition for skin external use for reducing wrinkle according to claim 1.

13. The cosmetic product according to claim 12, wherein the Anogeissus extract is an Anogeissus leiocarpus bark extract, the Punica extract is a Punica granatum extract, and the Palmae extract is a euterpe oleracea fruit extract.

14. The cosmetic product according to claim 13, further comprising, in parts by weight, 1-5 parts of a dictyophora indusiata extract.

15. The cosmetic product according to claim 14, comprising, in parts by weight, 18-20 parts of the Anogeissus leiocarpus bark extract, 11-14 parts of the Punica granatum extract, 5-7 parts of the euterpe oleracea fruit extract, 4-5 parts of the polygonatum odoratum extract, and 2-4 parts of the dictyophora indusiata extract.

16. The cosmetic product according to claim 12, wherein a method for preparing the Anogeissus extract comprises steps of:
pulverizing a Anogeissus bark and then sieving the same, then soaking the same by adding a first solvent; and performing ultrasonic extraction to a soaked mixture to obtain an extraction solution; and
filtering the extraction solution to obtain a filtrate, adding a second solvent to the filtrate, stirring and cooling the same, performing crystallization and precipitation to obtain a precipitate, and then drying the same to obtain the Anogeissus extract.

17. The cosmetic product according to claim 16, wherein a sieve used for the sieving has 70-100 meshes; the first solvent is dimethyl sulfoxide or N-methylpyrrolidone, a soaking time is 1-2.5 h, and a material-liquid ratio is 1:10-15 g/mL.

18. The cosmetic product according to claim 16, wherein an extraction temperature for the ultrasonic extraction is 70-90° C., an ultrasonic power is 250-450 W, and an extraction time is 15-35 min.

19. The cosmetic product according to claim 16, wherein the second solvent is one of methanol, ethanol and water, and a volume ratio of the second solvent to the filtrate is 1:2-5.

20. The cosmetic product according to claim 16, wherein a cooling rate is 2-4° C./s, and temperature of the crystallization and precipitation is 10-20° C.
